(19) United States
(12) Patent Application Publication
(10) Pub. No.: US 2026/0102324 A1
(43) Pub. Date: Apr. 16, 2026

(54) PERFUMING COMPOSITIONS COMPRISING A 2-(ALKYLSULFONYL)OCTAN-4-ONE

(71) Applicant: FIRMENICH SA, Satigny (CH)

(72) Inventors: Wolfgang FIEBER, Ornex (FR); Andreas HERRMANN, Arzier (CH)

(73) Assignee: FIRMENICH SA, Satigny (CH)

(21) Appl. No.: 19/116,658

(22) PCT Filed: Oct. 5, 2023
(86) PCT No.: PCT/EP2023/077527

§ 371 Date: Mar. 28, 2025

(30) Foreign Application Priority Data
Oct. 7, 2022 (EP) 22200355.0

(51) Int. Cl. A61K 8/46; A61Q 13/00

(52) U.S. Cl. CPC A61K 8/46; A61Q 13/00

(57) ABSTRACT

The present invention relates to a perfuming composition comprising a 2-(alkylsulfonyl)octan-4-one compound of formula (I) and an oil phase comprising at least one perfumery raw material, wherein the compound of formula (I) is present in the perfuming composition in an amount of at least 5 wt. %. The present invention further relates to a method for preparing the perfuming composition as well as to a consumer product comprising the perfuming composition.

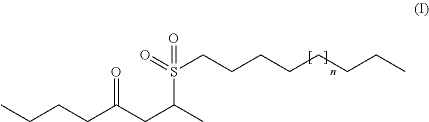

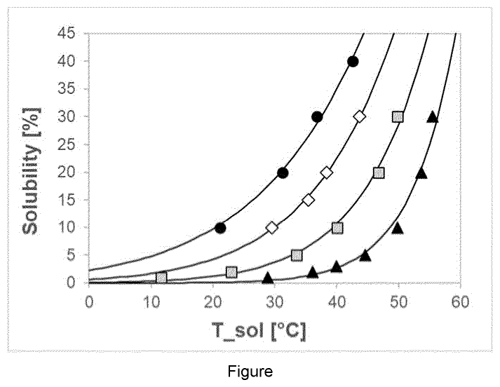

### TECHNICAL FIELD

The present invention relates to a perfuming composition comprising a 2-(alkylsulfonyl)octan-4-one compound of formula (I) and an oil phase comprising a perfumery raw material, wherein the compound of formula (I) is present in the perfuming composition in an amount of at least 5 wt. %. The present invention further relates to a method for preparing the perfuming composition as well as to a consumer product comprising the perfuming composition.

### BACKGROUND OF THE INVENTION

Consumers often correlate the efficiency of perfumed articles with the long-lastingness or substantivity of perfume perception. Perfumes are composed of a multitude of different volatile compounds, which are applied to a surface from which they evaporate to be smelled. The perfume is applied to surfaces, such as hard surfaces, fabric, skin or hair, via a perfume composition or a perfumed consumer article, as for example fine fragrances or diverse washing and cleaning agents. Due to the high volatility of the fragrances, which constitute perfumes, the odor emitted from the perfumed surface can only be perceived over a limited period of time. In particular, the so-called top-notes of a perfume evaporate quite rapidly. They are the most volatile compounds of the composition and represent the freshness of a perfume. Top-notes typically comprise, among others, citrus, flowery, green and fruity notes, and especially the fruity notes are well appreciated by consumers. Several classes of fruity notes are used in perfumes, one example are notes resembling of red berries, such as raspberries or strawberries. Oct-2-ene-4-one can be used as a perfumery ingredient to provide a natural strawberry odor when used in a perfume composition or a perfumed consumer article. However, the compound is quite volatile, and its odor imparted to surfaces, such as hard surfaces, fabric, skin or hair, only lasts for a relatively short period of time.

Consumers seek for fragrances that are stable in the targeted application and at the same time long-lasting and/or substantive to be smelled for several hours or even days after application. In particular, long-lasting red berry notes, such as e.g. strawberry notes, are desirable.

For this purpose, pro-fragrances such as 2-(alkylsulfonyl)octan-4-one compounds can be used that are able to slowly generate oct-2-en-4-one over time and thus provide a long-lasting strawberry odor to the environment (see WO 2019/243369).

2-(Alkylsulfonyl)octan-4-one compounds can be advantageously used in a perfuming composition, such as fragrance oils. However, this class of compounds cannot always be readily dissolved in fragrance oils. As an example, 2-(dodecylsulfonyl)octan-4-one is solid at room temperature and therefore the handling of this compound is different from that of a typical perfumery raw material. Melting the compound above its melting point (49-51° C.) prior to mixing with the fragrance oil is one option; however, 2-(dodecylsulfonyl)octan-4-one tends to resolidify quickly once the temperature drops below the melting point. Another option is directly combining the solid 2-(alkyldodecylsulfonyl)octan-4-one with the fragrance oil, which requires efficient stirring and/or heating for homogeneous mixing and full solubilization.

To overcome the issues mentioned above, 2-(alkylsulfonyl)octan-4-one compounds may be delivered in the form of a solution, i.e. the 2-(alkylsulfonyl)octan-4-one compounds may be dissolved in a solvent.

However, when the 2-(alkylsulfonyl)octan-4-one compounds are dissolved first into a solvent before being added to a perfuming composition, the compounds are provided in diluted form, which inevitably decreases the added amount of said compounds. In order to at least partially reduce this impact of dilution, high concentrations of 2-(alkylsulfonyl)octan-4-one compounds in the solution are desirable. To ensure a sufficiently high olfactive impact of the 2-(alkylsulfonyl)octan-4-one compounds in the final application, the perfuming composition should comprise the 2-(alkylsulfonyl)octan-4-one compounds at a level of at least 5 wt %

However, the inventors of the present invention found that that the provision of a solvent system that meets the above-mentioned requirements is not trivial. In particular, the inventors of the present invention found that solvents that are commonly used in the art to dissolve fragrances and pro-fragrances, such as dipropylene glycol (DIPG) or isopropyl myristate (IPM), are not capable of fully dissolving 2-(alkylsulfonyl)octan-4-one compounds in the required concentrations at room temperature. Hence, the objective of the present invention was to find solvents that are capable of dissolving high amounts of 2-(alkylsulfonyl)octan-4-one compounds at room temperature to ensure that olfactively sufficient amounts of 2-(alkylsulfonyl)octan-4-one compounds can be used in a perfuming composition.

### BRIEF DESCRIPTION OF THE FIGURE

Solubility of 2-(dodecylsulfonyl)octan-4-one in various liquid carriers and oil phases as a function of temperature. Black triangle: DIPG, grey squares: IPM, open diamonds: Hedione®, black circles: gamma-nonalactone and Cetiol® Ultimate (80/20). The continuous lines represent exponential functions fit to the experimental data.

### SUMMARY

The present invention relates to a perfuming composition comprising:
a 2-(alkylsulfonyl)octan-4-one compound of formula

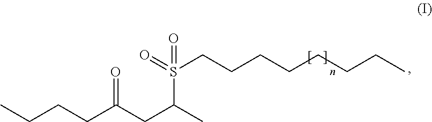

wherein n is an integer between 1 and 9;
an oil phase comprising at least one perfumery raw material;
wherein the compound of formula (I) is present in the perfuming composition in an amount of at least 5 wt. %.

Another aspect of the present invention relates to a method for preparing a perfuming composition according to the invention comprising the step of dissolving a 2-(alkylsulfonyl)octan-4-one compound of formula (I) in an oil phase comprising at least one perfumery raw material.

The present invention further relates to the use of at least one perfumery raw material to dissolve a 2-(alkylsulfonyl)octan-4-one compound of formula (I).

Moreover, the present invention relates to a consumer product comprising the perfuming composition according to the invention.

### DETAILED DESCRIPTION

The present invention relates to a perfuming composition comprising:
a 2-(alkyisuifonyl)octan-4-one compound of formula

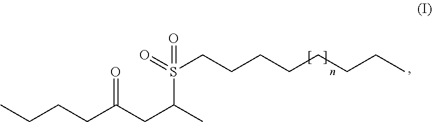

wherein n is an integer between 1 and 9;
an oil phase comprising at least one perfumery raw material;
wherein the compound of formula (I) is present in the perfuming composition in an amount of at least 5 wt. %.

By “oil phase” it is meant an ingredient or a composition that is a liquid at about 20° C. and that it is not or only to a small extent miscible with an aqueous phase.

In a particular embodiment, the perfuming composition consists of the compound of formula (I) and the oil phase.

According to the invention, the perfuming composition comprises a 2-(alkylsulfonyl)octan-4-one compound of formula (I).

Compounds according to formula (I) can act as pro-fragrance compounds in that they are able to slowly generate oct-2-en-4-one over time and thus are able to provide a long-lasting strawberry odor to the environment. The expression “strawberry odor” or “strawberry note” is understood as an odor reminding of a strawberry fruit.

The compounds according to formula (I) themselves are non-volatile and essentially odorless. The generation of oct-2-en-4-one from the compounds of formula (I) may be triggered by one or more of the following triggers: the presence of moisture/oxygen, pH changes, presence of enzymes, and increased temperatures.

According to the invention, the integer n in formula (I) is from 1 to 9.

In a particular embodiment, n is 1. In another embodiment, n is 2. In another embodiment, n is 3. In another embodiment, n is 4. In another embodiment, n is 5. In another embodiment, n is 6. In another embodiment, n is 7. In another embodiment, n is 8. In another embodiment, n is 9.

In a preferred embodiment, the integer n is from 3 to 7. Preferably, the integer n is from 4 to 6. More preferably, n is 5. In case that n is 5, the compound of formula (I) is 2-(dodecylsulfonyl)octan-4-one. Hence, in a preferred embodiment, the compound of formula (I) is 2-(dodecylsulfonyl)octan-4-one.

In a particular embodiment, the perfuming composition comprises more than one compound according to formula (I).

In an embodiment, the perfuming composition comprises 1 to 5 compounds of formula (I).

In an embodiment, the perfuming composition comprises one compound of formula (I).

In an embodiment, the perfuming composition comprises two compounds of formula (I).

In an embodiment, the perfuming composition comprises three compounds of formula (I).

In an embodiment, the perfuming composition comprises four compounds of formula (I).

In an embodiment, the perfuming composition comprises five compounds of formula (I).

According to the invention, the perfuming composition comprises an oil phase comprising at least one perfumery raw material.

In a preferred embodiment, the compound of formula (I) is fully dissolved in the oil phase at a temperature of 25° C. Hence, in said preferred embodiment, the oil phase is capable of fully dissolving the compound of formula (I) at a temperature of 25° C.

A skilled person is well aware of suitable means how to determine whether a compound of formula (I) is fully dissolved in the oil phase or not. For example, alight beam may be passed through a sample of dissolved compound at 25° C. In case that 100% light transparency is achieved, it can be assumed that the compound of formula (I) is fully dissolved in the oil phase at 25° C. The light beam may be based on LED light. Hence, in a particular embodiment, full solubility of the compound of formula (I) is determined based on full light transparency at 25° C. For the measurement, the dissolved sample may be present in a glass vial.

The term “solubility at 25° C.” is meant to represent the thermodynamic, equilibrium solubility of a compound of formula (I) at 25° C. A skilled person in the field is well aware that the solubility of any solid compound can be significantly increased with increasing temperature of the solvent system. Subsequently, even once the temperature is brought back to a lower temperature, for example to 25° C., the solid compound can still remain in solution for a certain amount of time while being supersaturated. However, this is an apparent solubility due to a non-equilibrium, metastable state with limited shelf-life stability, and therefore has to be distinguished from thermodynamic solubility at equilibrium. Thermodynamic solubility can for example be determined with the so-called shake flask method at a controlled temperature. Alternatively, it can be determined by slowly heating the compound and recording the solubilization temperature after complete dissolution.

In a particular embodiment, the oil phase is capable of fully dissolving the compound of formula (I).

In a particular embodiment, the oil phase consists of at least one perfumery raw material.

In a particular embodiment, the perfuming composition comprises the oil phase in an amount of 95 wt. % or less. In an embodiment, the perfuming composition comprises the oil phase in an amount of 90 wt. % or less, 80 wt. % or less, 70 wt. % or less, 60 wt. % or less, or 50 wt. % or less.

In a particular embodiment, the perfuming composition comprises the oil phase in an amount of from 50 to 95 wt. %. For example, from 60 to 95 wt. %, or 70 to 95 wt. %, or 80 to 95 wt. %, or 85 to 95 wt. %.

According to the invention, the oil phase comprises a perfumery raw material.

A “perfume compound” or “perfumery raw material (PRM)” is a compound, which is used as an active ingredient in perfuming compositions in order to impart a hedonic effect. In other words, a compound to be considered as being a perfume compound must be recognized by a skilled person in the art of perfumery as being able to impart or modify the odor of a composition in a positive or pleasant way, and not just as having an odor.

The nature and type of the perfumery raw material present in the invention's perfuming composition do not warrant a more detailed description here, which in any case would not be exhaustive, the skilled person being able to select them on the basis of his general knowledge and according to the intended use or application and the desired organoleptic effect. In general terms, these perfumery raw materials belong to chemical classes as varied as alcohols, lactones, aldehydes, ketones, esters, ethers, acetates, nitriles, thiols, terpenoids, nitrogenous or sulphureous heterocyclic compounds and essential oils, and perfumery raw materials can be of natural or synthetic origin.

Many of these perfuming ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, Van Nostrand Co., Inc., as well as in the abundant patent literature in the field of perfumery.

Perfumery raw materials may be, for example:
Aldehydic ingredients: (2E)-2-benzylideneoctanal, 4-methoxybenzaldehyde, 3-ethoxy-4-hydroxybenzaldehyde, 3,7-dimethyl-2,6-octadienal, 2,6-dimethylhept-5-enal, (E)-2-pentyl-3-phenyl-2-propenal, 4-hydroxy-3-methoxybenzaldehyde, benzo[d][1,3]dioxole-5-carbaldehyde, benzaldehyde, (E)-3-phenyl-2-propenal, 3-(2/4-ethylphenyl)-2,2-dimethylpropanal, (2E)-2-methyl-3-phenyl-2-propenal, (2E)-2-benzylideneoctanal, (+/−)-1-methyl-4-(4-methyl-3-pentenyl)-3-cyclohexene-1-carbaldehyde, (2E)-2-dodecenal, (2E,6Z)-2,6-nonadienal, 4-(2-propanyl)benzaldehyde, 4-methylbenzaldehyde, 4-formyl-2-methoxyphenyl 2-methylpropanoate, (3,4-dimethoxybenzylidene)(methyl)-λ3-oxidane, (2E,6Z)-2,6-nonadienal, (E)-2-decenal, (E)-2-tridecenal, 3,6,7-trimethyl-2,6-octadienal, decanal, 10-undecenal, 2-methylundecanal, and 2-hydroxybenzaldehyde;
Aromatic-herbal ingredients: camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionone isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate, 4-(4-hydroxyphenyl)butan-2-one, oct-2-en-4-one, 2,5-dimethyl-4-hydroxy-2H-furan-3-one, ethyl 3-methyl-3-phenyloxirane-2-carboxylate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.02,7]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonane and/or 3-(3-isopropyl-1-phenyl)butanal.

In a particular embodiment, the perfumery raw material is capable of fully dissolving the 2-(alkylsulfonyl)octan-4-one compound according to formula (I) at 25° C.

In a particular embodiment, the oil phase consists of a perfumery raw material.

In a particular embodiment, the oil phase comprises more than one perfumery raw material. For example, the oil phase comprises 2 to 50 perfumery raw materials, or 2 to 20 perfumery raw materials, or 2 to 10 perfumery raw materials.

In a preferred embodiment, the perfumery raw material is selected from the group of lactones.

In a preferred embodiment, the perfumery raw material comprises lactones, methyl 2-(3-oxo-2-pentylcyclopentyl)acetate, or a mixture thereof. Preferably, the perfumery raw material is selected from the group consisting of lactones, methyl 2-(3-oxo-2-pentylcyclopentyl)acetate (Hedione®), and a mixture thereof.

In a preferred embodiment, the perfumery raw material comprises methyl 2-(3-oxo-2-pentylcyclopentyl)acetate (Hedione®), oct-2-en-4-one, a gamma-lactone, a delta-lactone, or any mixture thereof. Preferably, the gamma-lactone is gamma-nonalactone, gamma-decalactone, gamma-undecalactone, or gamma-valerolactone. Preferably, the delta-lactone is delta-nonalactone.

In a preferred embodiment, the perfumery raw material is selected from the group consisting of methyl 2-(3-oxo-2-pentylcyclopentyl)acetate (Hedione®), oct-2-en-4-one, a gamma-lactone, a delta-lactone, and a mixture thereof. Preferably, the gamma-lactone is gamma-nonalactone, gamma-decalactone, gamma-undecalactone, or gamma-valerolactone. Preferably, the delta-lactone is delta-nonalactone.

In a particular embodiment, the perfumery raw material is methyl 2-(3-oxo-2-pentylcyclopentyl)acetate (Hedione®).

In a particular embodiment, the perfumery raw material is oct-2-en-4-one. In view of the potential of the compound of formula (I) to release oct-2-en-4-one, said embodiment is particularly capable of providing an intense strawberry odor. Oct-2-ene-4-one can be in the form of its (E)- or (Z)-isomer, or a mixture thereof. Preferably, oct-2-en-4-one is mainly in the form of its (E)-isomer, containing a minor amount of the (Z)-form, even more preferably, oct-2-en-4-one is in the form of its (E)-isomer.

In a particular embodiment, the perfumery raw material is a gamma-lactone.

In a particular embodiment, the perfumery raw material is gamma-valerolactone.

In a particular embodiment, the perfumery raw material is gamma-nonalactone.

In a particular embodiment, the perfumery raw material is gamma-decalactone.

In a particular embodiment, the perfumery raw material is gamma-undecalactone.

In a particular embodiment, the perfumery raw material is a delta-lactone.

In a particular embodiment, the perfumery raw material is delta-nonalactone.

In a particular embodiment, the perfuming composition comprises at least 5 wt. % of lactones, oct-2-en-4-one, methyl 2-(3-oxo-2-pentylcyclopentyl)acetate, or a mixture thereof.

In a particular embodiment, the perfuming composition comprises at least 10 wt % of lactones, oct-2-en-4-one, methyl 2-(3-oxo-2-pentylcyclopentyl)acetate, or a mixture thereof.

In a particular embodiment, the perfuming composition comprises at least 15 wt % of lactones, oct-2-en-4-one, methyl 2-(3-oxo-2-pentylcyclopentyl)acetate, or a mixture thereof.

In a particular embodiment, the perfuming composition comprises at least 20 wt % of lactones, oct-2-en-4-one, methyl 2-(3-oxo-2-pentylcyclopentyl)acetate, or a mixture thereof.

In a particular embodiment, the perfuming composition comprises at least 25 wt % of lactones, oct-2-en-4-one, methyl 2-(3-oxo-2-pentylcyclopentyl)acetate, or a mixture thereof.

In a particular embodiment, the perfuming composition comprises at least 30 wt % of lactones, oct-2-en-4-one, methyl 2-(3-oxo-2-pentylcyclopentyl)acetate, or a mixture thereof.

In a particular embodiment, the perfuming composition comprises at most 95 wt. % of lactones, oct-2-en-4-one, methyl 2-(3-oxo-2-pentylcyclopentyl)acetate, or a mixture thereof.

In a particular embodiment, the perfuming composition comprises at most 90 wt. % of lactones, oct-2-en-4-one, methyl 2-(3-oxo-2-pentylcyclopentyl)acetate, or a mixture thereof.

In a particular embodiment, the perfuming composition comprises at most 85 wt. % of lactones, oct-2-en-4-one, methyl 2-(3-oxo-2-pentylcyclopentyl)acetate, or a mixture thereof.

In a particular embodiment, the perfuming composition comprises at most 80 wt. % of lactones, oct-2-en-4-one, methyl 2-(3-oxo-2-pentylcyclopentyl)acetate, or a mixture thereof.

In a particular embodiment, the perfuming composition comprises at most 75 wt. % of lactones, oct-2-en-4-one, methyl 2-(3-oxo-2-pentylcyclopentyl)acetate, or a mixture thereof.

In a particular embodiment, the perfuming composition comprises at most 70 wt. % of lactones, oct-2-en-4-one, methyl 2-(3-oxo-2-pentylcyclopentyl)acetate, or a mixture thereof.

In a particular embodiment, the perfumery raw material is liquid at 25° C.

In a preferred embodiment, the perfumery raw material is an essential oil.

In another embodiment, the oil phase comprises at least one perfumery raw material being an essential oil and at least one perfumery raw material not being an essential oil.

In a particular embodiment, the essential oil is selected from the group consisting of eucalyptus oil, orange oil, and patchouli oil.

In a particular embodiment, the oil phase comprises more than one essential oil. For example, the oil phase comprises 2 to 50 oils, or 2 to 20 oils, or 2 to 10 oils.

In a particular embodiment, the perfuming composition comprises the essential oil in an amount of 95 wt. % or less. In an embodiment, the perfuming composition comprises the oil in an amount of 90 wt. % or less, 80 wt. % or less, 70 wt. % or less, 60 wt. % or less, or 50 wt. % or less.

In a particular embodiment, the perfuming composition comprises the perfumery raw material in an amount of 95 wt. % or less. In an embodiment, the perfuming composition comprises the perfumery raw material in an amount of 90 wt. % or less, 80 wt. % or less, 70 wt. % or less, 60 wt. % or less, or 50 wt. % or less.

In a particular embodiment, the perfuming composition comprises the perfumery raw material in an amount of from 50 to 95 wt. %. For example, from 60 to 95 wt. %, or 70 to 95 wt. %, or 80 to 95 wt. %, or 85 to 95 wt. %.

In a particular embodiment, the oil phase further comprises a liquid carrier.

In a particular embodiment, the liquid carrier may be selected from the group consisting of diethyl phthalate, dimethyl glutarate, dimethyl adipate, ethyl oleate, dipropylene glycol (DIPG), DIPG methyl ether acetate, DIPG monomethyl ether, DIPG monoethyl ether, DIPG n-propyl ether, heptyl undecylenate, isocetyl alcohol, neopentyl glycol diethylhexanoate, isopropyl myristate (IPM), triacetin, dioctyl adipate, butoxypropanol, benzyl benzoate, benzyl alcohol, medium chain triglycerides, isopropylidene glycerol, ethyl lactate, diethylene glycol methyl ether, triethyl acetate, castor oil, 3-methoxy-3-methyl butanol, tripropylene glycol methyl ether, propylene glycol methyl ether, hexylene glycol, pentylene glycol, butylene glycol, 1,3-butanediol, 2,3-butanediol, 2-methyl butanol, n-propanol, iso-propanol, ethanol, propylene glycol, 1,3-propanediol, glycerol, n-alkanes and iso-alkanes.

In a particular embodiment, the liquid carrier may be one or more of the group of alkanes, such as undecane or tridecane.

In a particular embodiment, the oil phase comprises a lactone and a liquid carrier selected from the group of IPM and alkanes.

In a particular embodiment, the oil phase does not comprise more than 50 wt. % of a liquid carrier preferably not more than 20 wt. %, more preferably not more than 10 wt. %.

In a particular embodiment, the oil phase does not comprise more than 50 wt. % of dipropylene glycol (DIPG), preferably not more than 20 wt. %, more preferably not more than 10 wt. %.

In a particular embodiment, the oil phase does not comprise one or more of liquid carriers selected from the group consisting of diethyl phthalate, dimethyl glutarate, dimethyl adipate, ethyl oleate, dipropylene glycol (DIPG), DIPG methyl ether acetate, DIPG monomethyl ether, DIPG monoethyl ether, DIPG n-propyl ether, heptyl undecylenate, isocetyl alcohol, neopentyl glycol diethylhexanoate, isopropyl myristate (IPM), triacetin, dioctyl adipate, butoxypropanol, benzyl benzoate, benzyl alcohol, medium chain triglycerides, isopropylidene glycerol, ethyl lactate, diethylene glycol methyl ether, triethyl acetate, castor oil, 3-methoxy-3-methyl butanol, tripropylene glycol methyl ether, propylene glycol methyl ether, hexylene glycol, pentylene glycol, butylene glycol, 1,3-butanediol, 2,3-butanediol, 2-methyl butanol, n-propanol, iso-propanol, ethanol, propylene glycol, 1,3-propanediol, glycerol, n-alkanes and iso-alkanes.

In a particular embodiment, the oil phase does not comprise dipropylene glycol (DIPG).

In a particular embodiment, the oil phase does not comprise isopropyl myristate (IPM).

In a particular embodiment, the oil phase does not comprise triethyl citrate.

According to the invention, the perfuming composition comprises the compound of formula (I) in an amount of at least 5 wt. %.

The inventors of the present invention surprisingly found that it is difficult to find an oil phase that is capable of fully dissolving the compounds of formula (I) at high concentrations at 25° C. (room temperature). In particular, it has been found that it is difficult to find solvents/liquid carriers that are capable of fully dissolving the compounds of formula (I) at amounts of at least 5 wt. % and that are suitable to be used for perfume compositions. The requirement that solvents need to be suitable for perfume composition significantly limits the spectrum of potentially useful solvents for dissolving compounds of formula (I). For example, some solvents cannot be used for regulatory reasons, or because they have a strong chemical odor that is not compatible with perfumery applications. Specifically, 2-methyl-tetrahydrofurane may be considered a good solvent for compounds of formula (I), but it is not suitable for perfume compositions in view of its regulatory status.

The claimed levels of compounds of formula (I) in the perfuming composition are beneficial to ensure that a significant impact can be achieved by the compounds of formula (I) when the perfuming composition is added to a consumer product. In this regard, the inventors found that solvents that are commonly used for the preparation of perfuming compositions are less suitable for fully dissolving the compounds of formula (I) at the claimed concentrations, but rather it has been surprisingly found that perfumery raw materials are suitable for dissolving the compounds of formula (I) at high concentrations at 25° C.

In a particular embodiment, the compound of formula (I) is present in the perfuming composition in an amount of from 5 to 50 wt. %, preferably 5 to 20 wt. %, more preferably 5 to 15 wt. %.

In a particular embodiment, the compound of formula (I) is present in the perfuming composition in an amount of at least 5 wt. %.

In a particular embodiment, the compound of formula (I) is present in the perfuming composition in an amount of at least 10 wt %.

In a particular embodiment, the compound of formula (I) is present in the perfuming composition in an amount of at least 15 wt %.

In a particular embodiment, the compound of formula (I) is present in the perfuming composition in an amount of at least 20 wt %.

In a particular embodiment, the compound of formula (I) is present in the perfuming composition in an amount of at least 25 wt %.

In a particular embodiment, the compound of formula (I) is present in the perfuming composition in an amount of at least 30 wt %.

In a particular embodiment, the perfuming composition further comprises a perfumery adjuvant.

By “perfumery adjuvant” an ingredient is meant that is capable of imparting additional added benefit such as a color, a particular light resistance, chemical stability, etc. A detailed description of the nature and type of adjuvant commonly used in perfuming compositions cannot be exhaustive, but it has to be mentioned that said ingredients are well known to a person skilled in the art. However, one may cite as specific non-limiting examples the following: viscosity agents (e.g. surfactants, thickeners, gelling and/or rheology modifiers), stabilizing agents (e.g. preservatives, antioxidants, heat/light and or buffers or chelating agents, such as BHT), coloring agents (e.g. dyes and/or pigments), preservatives (e.g. antibacterial or antimicrobial or antifungal or anti-irritant agents), abrasives, skin cooling agents, fixatives, insect repellants, ointments, vitamins, and mixtures thereof.

In another aspect, the present invention relates to a method for preparing a perfuming composition according to the invention comprising the step of dissolving a 2-(alkylsulfonyl)octan-4-one compound of formula

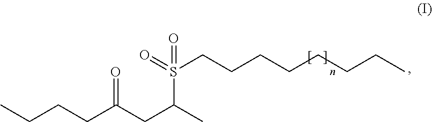

in an oil phase comprising at least one perfumery raw material.

In a particular embodiment, the 2-(alkylsulfonyl)octan-4-one compound of formula (I) is fully dissolved in the oil phase at a temperature of 25° C.

The embodiments and modifications given above for the perfuming composition according to the invention also apply to the method according to the invention.

Another aspect of the present invention relates to the use of at least one perfumery raw material to dissolve a 2-(alkylsulfonyl)octan-4-one compound of formula

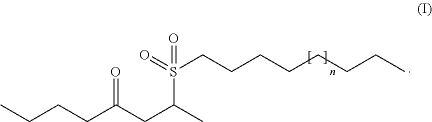

In a particular embodiment, the 2-(alkylsulfonyl)octan-4-one compound of formula (I) is dissolved in the perfumery raw material and/or an oil at a concentration of at least 5 wt. %.

In a particular embodiment, the 2-(alkylsulfonyl)octan-4-one compound of formula (I) is fully dissolved at a temperature of 25° C.

In a particular embodiment, the 2-(alkylsulfonyl)octan-4-one compound of formula (I) is present in a perfuming composition.

The invention's perfuming composition can be advantageously used in all the fields of modern perfumery, i.e. fine or functional perfumery, to positively impart or modify the odor of a consumer product into which said composition is added.

Consequently, another aspect of the invention concerns a consumer product comprising the perfuming composition according to the invention.

For the sake of clarity, it is mentioned that the term “consumer product” is understood as a consumer product, which is expected to deliver at least a pleasant perfuming effect to the surface to which it is applied (e.g. skin, hair, textile, or hard surface). For the sake of clarity, the consumer product is a non-edible product.

The nature and type of the constituents of the perfumed consumer product do not warrant a more detailed description here, which in any case would not be exhaustive, the skilled person being able to select them based on his general knowledge and according to the nature and the desired effect of the product.

Non-limiting examples of suitable consumer products include a perfume, such as a fine perfume, a splash or eau de parfum, a cologne or a shave or after-shave lotion; a fabric care product, such as liquid, a pod or solid detergent optionally in the form of a pod or or tablet, a fabric softener, a liquid or solid scent booster, a dryer-sheet, a fabric refresher, an ironing water, a paper, a bleach, a carpet cleaner, a curtain-care product; a body-care product, such as a hair care product (e.g. a shampoo, a leave-on or rinse-off hair conditioner, a coloring preparation or a hair spray, a color-care product, a hair shaping product), a dental care product, a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furniture care product, a wipe, a dish detergent or a hard-surface (e.g. a floor, bath, sanitary or a window-cleaning) cleaner; a leather care product; a car care product, such as a polish, a wax or a plastic cleaner.

In a particular embodiment, the consumer product is a personal care product or home care product, preferably a fabric conditioner, a solid or liquid detergent, a hard surface cleaner, a shampoo, a shower gel, a leave-on conditioner, or a rinse-off conditioner.

In a particular embodiment, the consumer product has an acidic pH value, preferably a pH below 5.5, preferably from 2.5 to 5.5.

The proportions in which the perfuming composition according to the invention can be incorporated into the various aforementioned articles or compositions vary within a wide range of values. These values are dependent upon the nature of the article or product to be perfumed.

In a particular embodiment, the consumer product comprises the perfuming composition in an amount of from 0.1 to 10 wt. %, preferably of from 0.2 to 5 wt. %, more preferably from 0.3 to 4 wt. %, even more preferably from 0.4 to 3 wt. % based on the total weight of the consumer product.

In a particular embodiment, the consumer product comprises the perfuming composition in an amount of less than 1 wt. %.

In a particular embodiment, the consumer product comprises the perfuming composition in an amount of higher than 2 wt. %.

In a particular embodiment, the consumer product comprises the perfuming composition in an amount of less than 1 wt. % or in an amount of higher than 2 wt. %.

In a particular embodiment, the consumer product comprises the perfuming composition in an amount of 0.1 wt. % to less than 1 wt. %.

In a particular embodiment, the consumer product comprises the perfuming composition in an amount of higher than 2 wt. % to 10 wt. %.

In a particular embodiment, the consumer product is a perfume, a fabric care product, a body-care product, a cosmetic preparation, a skin-care product, an air care product or a home care product. Preferably, the consumer product is a fabric softener, a shower gel or a rinse-off hair conditioner.

According to a particular embodiment of the invention, the invention's perfumed consumer product is a liquid fabric softener comprising a fabric softener active base in amount comprised between 85 and 100% by weight, based on the total weight of the perfumed consumer product. The fabric softener active base may comprise dialkyl quaternary ammonium salts, dialkyl ester quaternary ammonium salts, Hamburg esterquat, triethanolamine quat, silicones and mixtures thereof.

According to a particular embodiment of the invention, the invention's consumer product is an all-purpose cleaner comprising an all-purpose cleaner active base in amount comprised between 85 and 100% by weight, based on the total weight of the consumer product. The all-purpose active base may comprise linear alkylbenzene sulfonates (LAS) in an amount comprised between 0 and 4%, preferably 1 and 2%, nonionic surfactant in an amount comprised between 0 and 8%, preferably 2 and 4% and acid such as citric acid in an amount comprised between 0.1 and 0.5%.

According to a particular embodiment of the invention, the invention's perfumed consumer product is shampoo or a shower gel comprising a shampoo or shower gel active base in amount comprised between 85 and 100% by weight, based on the total weight of the perfumed consumer product. The shampoo or shower gel active base may comprise sodium alkylether sulfates, ammonium alkylether sulfates, alkylamphoacetates, cocamidopropyl betaine, cocamide MEA, alkyl polyglucosides and amino acid-based surfactants.

According to a particular embodiment of the invention, the invention's perfumed consumer product is a soap bar comprising a soap active base in amount comprised between 85 and 100% by weight, based on the total weight of the perfumed consumer product. The soap bar active base may comprise salt of a weak acid, typically, a salt of weak acid, which may be a fatty acid and strong base like sodium hydroxide.

According to a particular embodiment of the invention, the invention's perfumed consumer product is a rinse-off conditioner comprising a rinse-off conditioner active base in amount comprised between 85 and 99.95% by weight, based on the total weight of the perfumed consumer product. The rinse-off conditioner active base may comprise cetyltrimonium chloride, stearyl trimonium chloride, benzalkonium chloride, behentrimonium chloride and mixture thereof.

According to a particular embodiment of the invention, the invention's consumer product is a liquid detergent comprising liquid detergent active base in amount comprised between 85 and 100% by weight, based on the total weight of the consumer product. The liquid detergent active base may comprise anionic surfactants such as alkylbenzene sulfonates (ABS), linear alkylbenzene sulfonates (LAS), secondary alkyl sulfonates (SAS), primary alcohol sulfates (PAS), lauryl ether sulfates (LES), sodium lauryl ether sulfates (SLES), methyl ester sulfonates (MES); nonionic surfactants such as alkyl amines, alkanolamides, fatty alcohol ethoxylates (FAE), ethylene oxide (EO) and propylene oxide (PO) copolymers, amine oxides, alkyl polyglucosides, alkyl polyglucosamides; or mixtures thereof.

According to a particular embodiment of the invention, the invention's consumer product is a solid detergent comprising a solid detergent active base in amount comprised between 85 and 100% by weight, based on the total weight of the consumer product. The solid detergent active base may comprise at least one surfactant chosen in the group consisting of anionic, nonionic, cationic, zwitterionic surfactant and mixtures thereof. The surfactant in the solid detergent active base is preferably chosen in the group consisting of linear alkene benzene sulfonates (LABS), sodium laureth sulfate, sodium lauryl ether sulfates (SLES), sodium lauryl sulfate (SLS), alpha olefin sulfonates (AOS), methyl ester sulfonates (MES), alkyl polyglycosides (APG), primary alcohol ethoxylates and in particular lauryl alcohol ethoxylates (LAE), primary alcohol sulfonates (PAS), soap and mixtures thereof. The solid detergent active base may comprise a further component, commonly used in powder detergent consumer product, selected from the group consisting of bleaching agents such as EDTA (tetraacetylethylenediamine); buffering agent; builders such as zeolites, sodium carbonate or mixture thereof; soil release or soil suspension polymers; granulated enzyme particles such as cellulase, lipase, protease, mannanase, pectinase or mixtures thereof; corrosion inhibitor; antifoaming; dyes; fillers such as sodium silicate, sodium sulfate or mixture thereof; source of hydrogen peroxide such as sodium percarbonate or sodium perborate; and mixtures thereof.

In a particular embodiment, the consumer product comprises a preservative, preferably selected from the group consisting of benzisothiazolin-3-one, methylchloroisothiazolinone, methylisothiazolinone, and any mixture thereof.

The present invention also relates to the use of the perfuming composition according to the invention for the preparation of a consumer product.

### Examples

### Solubility Measurements:

Solid 2-(dodecylsulfonyl)octan-4-one was mixed with different solvents and transferred to a 8 mL vial equipped with an overhead stirrer. The sample was placed in a Crystalline PV instrument (Technobis, The Netherlands). Heating and cooling steps were applied under mechanical stirring, and the samples were monitored by following transparency via a LED light beam that passes through the vial. Full (100%) transparency is attained when 2-(dodecylsulfonyl)octan-4-one is completely dissolved in the solvent. On the other hand, when the measured values of transparency are below 100% the dissolution process is still incomplete.

### Determination of the Solubilization Temperature:

A first heating step was carried out by quickly (+5° C./min) heating the sample above its solubility temperature to 60° C. After complete dissolution, a cooling ramp to 1° C. was applied (−2° C./min), i.e. below the solubility temperature of the sample, where the sample was kept stirring until 2-(dodecylsulfonyl)octan-4-one has crystallized. Subsequently a second heating step was carried out by applying a shallow heating ramp (+0.1° C./min) in order to dissolve the material under quasi-isothermal conditions. The transparency of the sample was monitored as a function of time during the heating ramp. Once the signal has attained 100% transparency, the dissolution process was complete and the respective temperature was taken as the solubilization temperature (T_sol).

### Solubilization Temperatures of 5 wt. % of 2-(Dodecylsulfonyl)Octan-4-One in Different Solvents/Perfumery Raw Materials

Following the methodology described above, 2-(dodecylsulfonyl)octan-4-one was mixed with various solvents/liquid carriers that are suitable for perfumery compositions or with perfumery raw materials at a concentration of 5 wt. % and the solubilization temperature has been determined. The solvents and perfumery raw materials used and the respective solubilization temperatures are depicted in Tables 1-3 below.

TABLE 1
Solubilization temperatures of 5 wt. % of 2-(dodecylsulfonyl)octan-
4-one in differentperfumery raw materials.

| | | | Soluble | |
|---|---|---|---|---|
| | Concentration of 2- | | after | |
| Oil phase comprising | (dodecylsulfonyl) | Soluble | cooling to | |
| PRM | octan-4-one [wt. %] | at 60° C. | 25° C. | T_sol [° C.] |
| gamma-Decalactone | 5 | yes | yes | 14.6 |
| gamma-Undecalactone | 5 | yes | yes | 15.3 |
| gamma-Nonalactone/ | 5 | yes | yes | 10.5 |
| Cetiol ® Ultimate¹ (80/20; | | | | |
| w/w) | | | | |
| Hedione ®² | 5 | yes | yes | 21.0 |
| gamma-Valerolactone | 5 | yes | yes | 23.0 |
| gamma-Valerolactone/ | 5 | yes | yes | 15.3 |
| Isopropyl Myristate (IPM) | | | | |
| (70/30 w/w) | | | | |

¹Cetiol ® Ultimate: mixture of undecane and tridecane
²Hedione ®: methyl 2-(3-oxo-2-pentylcyclopentyl)acetate

TABLE 2
Solubilization temperatures of 5 wt. % of 2-(dodecylsulfonyl)octan-
4-one in different fragrance compositions.

| | | | Soluble | |
|---|---|---|---|---|
| | Concentration of 2- | | after | |
| Oil phase comprising | (dodecylsulfonyl) | Soluble at | cooling to | |
| PRM | octan-4-one [wt. %] | 60° C. | 25° C. | T_sol [° C.] |
| Fragrance composition F1 | 5 | yes | yes | 21.1 |
| Fragrance composition F2 | 5 | yes | yes | 19.8 |
| Fragrance composition F3 | 5 | yes | yes | 17.8 |
| Fragrance composition F4 | 5 | yes | yes | 24.0 |
| Fragrance composition F5 | 5 | yes | yes | 16.3 |

TABLE 3
Solubilization temperatures of 5 wt. % of 2-(dodecylsulfonyl)octan-4-one in
different solvents/liquid carriers compatible with perfumery applications.

| | Concentration of 2- | | Soluble after | |
|---|---|---|---|---|
| Oil phase not comprising | (dodecylsulfonyl) | Soluble at | cooling to | |
| PRM | octan-4-one [wt. %] | 60° C. | 25° C. | T_sol [° C.] |
| Ethyl lactate | 5 | yes | no | 26.5 |
| Dimethylglutarate (DBE-5) | 5 | yes | no | 27.3 |
| Isopropyl myristate (IPM) | 5 | yes | no | 33.5 |
| Triethyl citrate | 5 | yes | no | 36.2 |
| Triacetine | 5 | yes | no | 41.1 |
| Dipropylene glycol (DIPG) | 5 | yes | no | 44.6 |
| Cetiol ® Ultimate | 5 | yes | no | 45.4 |

The fragrance compositions given in Table 2 were as follows:

TABLE 4
Composition of fragrance composition F1

| PRM | wt % |
|---|---|
| Phenylethyl alcohol | 26.7 |
| Benzyl acetate | 8 |
| Florol ®¹ | 16 |
| Hedione ® | 13.3 |
| Dihydromyrcenol | 2.7 |
| Geraniol | 13.3 |
| Citronellol | 2.7 |
| Helvetolide ®² | 5.3 |
| Iso E Super³ | 2.7 |
| Habanolide ®⁴ | 8 |
| Muscenone ®⁵ | 1.3 |

¹Florol ®: 2-isobutyl-4-methyltetrahydro-2H-pyran-4-ol
²Helvetolide ®: 2-(1-(3,3-dimethylcyclohexyl)ethoxy)-2-methylpropyl propionate
³Iso E Super: 1-(octahydro-2,3,8,8-tetramethyl-2-naphthalenyl)-1-ethanone (mixture of isomers)
⁴Habanolide ®: oxacyclohexadec-12- and/or 13-en-2-one
⁵Muscenone ®: 3-methyl-4- or 5-cyclopentadecen-1-one (mixture of E/Z isomers)

TABLE 5
Composition of fragrance composition F2

| PRM | wt % |
|---|---|
| Benzyl acetate | 20 |
| Dihydromyrcenol | 20 |
| Coranol¹ | 10 |
| Neobutenone ®² | 0.1 |
| Helvetolide ® | 4 |
| Iso E Super | 26 |
| Ambrox ® | 2 |
| Exaltolide ® | 10 |
| DIPG | 7.9 |

¹Coranol: 4-cyclohexyl-2-methylbutan-2-ol
²Neobutenone ®: 1-(3,3- or 5,5-dimethylcyclohex-1-en-1-yl)pent-4-en-1-one
³Ambrox ®: 3a,6,6,9a-tetramethyldodecahydronaphtho[2,1-b]furan
⁴Exaltolide ®: oxacyclohexadecan-2-one

TABLE 6
Composition of fragrance composition F3

| PRM | wt % |
|---|---|
| Benzyl acetate | 21.7 |
| Dihydromyrcenol | 21.7 |
| Coranol | 10.9 |
| Neobutenone ® | 0.1 |
| Helvetolide ® | 4.3 |
| Iso E Super | 28.2 |
| Ambrox ® | 2.2 |
| Exaltolide ® | 10.9 |

TABLE 7
Composition of fragrance composition F4

| PRM | wt % |
|---|---|
| Hedione ® | 1 |
| Muscenone ® | 12 |
| Habanolide ® | 20 |
| Helvetolide ® | 6 |
| Ambrox ® | 1 |
| Exaltolide ® | 60 |

TABLE 8
Composition of fragrance composition F5

| PRM | wt % |
|---|---|
| gamma-Undecalactone | 40 |
| Hedione ® | 20 |
| Habanolide ® | 20 |
| Manzanate¹ | 10 |
| Fructalate ® | 10 |

¹Manzanate: ethyl 2-methylpentanoate
²Fructalate ®: diethyl cyclohexane-1,4-dicarboxylate

It can be observed from Tables 1-3 above that an oil phase comprising perfumery raw material and mixtures of perfumery raw materials were capable of fully dissolving 2-(dodecylsulfonyl)octan-4-one at a concentration level of 5 wt. % at 25° C. However, typical solvents used in perfumery (such as ethyl lactate, dimethylglutarate, isopropyl myristate, triethyl citrate, triacetine, dipropylene glycol, and Cetiol® Ultimate) were by themselves not capable of fully dissolving 2-(dodecylsulfonyl)octan-4-one at a concentration level of 5 wt. % at 25° C., as only at higher temperatures than 25° C. full solubility of 2-(dodecylsulfonyl)octan-4-one could be achieved for these solvents.

### Temperature Solubility Curves

Following the methodology described above, 2-(dodecylsulfonyl)octan-4-one was mixed with various solvents or perfumery raw materials at different concentrations, and the solubilization temperature has been determined.

TABLE 9
Solubilization temperatures of 2-(dodecylsulfonyl)octan-4-one at various
concentrations in different solvents/perfumery raw materials.

| | | | | gamma- | | gamma- |
|---|---|---|---|---|---|---|
| | | | | Nonalactone/ | | Valero- |
| Concentration of 2- | | | | Cetiol ® | gamma- | lactone/ |
| (dodecylsulfonyl) | | | | Ultimate¹ | Valero- | IPM (70/ |
| octan-4-one [wt. %] | DIPG | MIP | Hedione ® | (80/20; w/w) | lactone | 30 w/w) |
| 1 | 28.9 | 11.6 | | | | |
| 2 | 36.1 | 23.0 | | | | |
| 3 | 40.0 | | | | | |
| 5 | 44.6 | 33.5 | 21.0 | 10.5 | 23.0 | 15.3 |
| 10 | 49.8 | 40.1 | 29.5 | 21.2 | 30.5 | 23.6 |
| 15 | | | 35.4 | | | |
| 20 | 53.7 | 46.7 | 38.4 | 31.2 | 36.9 | 32.3 |
| 30 | 55.5 | 49.8 | 43.7 | 36.8 | 41.2 | 38.0 |
| 40 | | | | 42.5 | 43.5 | |

### Soluble Amounts of 2-(Dodecylsulfonyl)Octan-4-One in Different Solvents/Perfumery Raw Materials at 25° C.:

The solubility of 2-(dodecylsulfonyl)octan-4-one at 25° C. in various solvents or perfumery raw materials has been determined based on the data presented in Table 9. For each solvent or perfumery raw material, the measured solubility as a function of temperature has been fitted to an exponential function. The solubility at 25° C. has been obtained by extrapolation or interpolation of this exponential function. The results/exponential curves are shown in the Figure (black triangle: DIPG, grey squares: IPM, open diamonds: Hedione®, black circles: gamma-nonalactone and Cetiol® Ultimate (80/20); the continuous lines represent exponential functions fit to the experimental data).

TABLE 10
Fully dissolvable amounts of 2-(dodecylsulfonyl)octan-
4-one at 25° C. in perfumery raw materials, obtained
from extrapolation of temperature-solubility curves.

| Oil phase | Solubility of 2-(dodecylsulfonyl) |
|---|---|
| comprising PRM | octan-4-one at 25° C. [wt. %] |
| gamma-Nonalactone/ | 13.7 |
| Cetiol ® Ultimate | |
| (80/20 w/w) | |
| Hedione ® | 6.62 |
| gamma-Valerolactone | 5.5 |
| gamma-Valerolactone/ | 11.3 |
| IPM (70:30 w/w) | |

TABLE 11
Fully dissolvable amounts of 2-(dodecylsulfonyl)octan-
4-one at 25° C. in DIPG and IPM, obtained from
extrapolation of temperature-solubility curves.

| Oil phase not | Solubility of 2-(dodecylsulfonyl) |
|---|---|
| comprising PRM | octan-4-one at 25° C. [wt. %] |
| DIPG | 0.66 |
| IPM | 2.55 |

From Table 10 and 11, it can be observed that an oil phase comprising gamma-nonalactone and Cetiol® Ultimate (80/20; v/v), and Hedione®, respectively, was capable of fully dissolving high amounts of 2-(dodecylsulfonyl)octan-4-one (amounts higher than 5 wt. %). However, the common solvents isopropyl myristate and dipropylene glycol were not able to fully dissolve 2-(dodecylsulfonyl)octan-4-one at 25° C. in high amounts. Only amounts below 3 wt. % could be dissolved by these solvents at 25° C.

1. A perfuming composition comprising:
a 2-(alkylsulfonyl)octan-4-one compound of formula 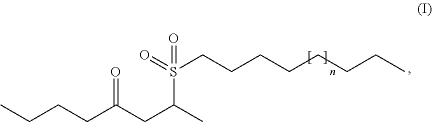
wherein n is an integer between 1 and 9;
an oil phase comprising at least one perfumery raw material;
wherein the compound of formula (I) is present in the perfuming composition in an amount of at least 5 wt. %.

2. The perfuming composition of claim 1, wherein the compound of formula (I) is fully dissolved in the oil phase at a temperature of 25° C.

3. The perfuming composition of claim 1, wherein the perfumery raw material is selected from the group consisting of fragrance oils, essential oils, and a combination thereof.

4. The perfuming composition of claim 1, wherein n is an integer between 3 and 7.

5. The perfuming composition of claim 1, wherein the perfuming composition consists of the compound of formula (I) and the oil phase.

6. The perfuming composition of claim 1, wherein the compound of formula (I) is present in the perfuming composition in an amount of from 5 to 50 wt. %.

7. The perfuming composition of claim 1, wherein the oil phase consists of at least one perfumery raw material.

8. The perfuming composition of claim 1, wherein the oil phase further comprises a solvent.

9. The perfuming composition of claim 1, wherein the perfumery raw material comprises lactones, methyl 2-(3-oxo-2-pentylcyclopentyl)acetate, or a mixture thereof.

10. The perfuming composition of claim 1, wherein the perfumery raw material is selected from the group consisting of methyl 2-(3-oxo-2-pentylcyclopentyl)acetate, oct-2-en-4-one, a gamma-lactone, a delta-lactone, and a mixture thereof.

11. The perfuming composition of claim 1, wherein the oil phase does not comprise a liquid carrier.

12. The perfuming composition of claim 1, wherein the oil phase does not comprise more than 50 wt. % of dipropylene glycol (DIPG).

13. A method for preparing a perfuming composition according to claim 1, the method comprising the step of dissolving a 2-(alkylsulfonyl)octan-4-one compound of formula 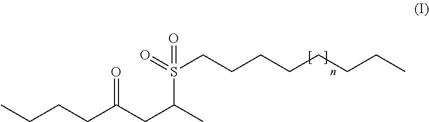
in an oil phase comprising at least one perfumery raw material.

14. (canceled)

15. A consumer product comprising the perfuming composition according to claim 1.

16. The perfuming composition of claim 4, wherein n is an integer between 4 and 6.

17. The perfuming composition of claim 16, wherein n is 5.

18. The perfuming composition of claim 6, wherein the compound of formula (I) is present in the perfuming composition in an amount of from 5 to 20 wt. %.

19. The perfuming composition of claim 18, wherein the compound of formula (I) is present in the perfuming composition in an amount of from 5 to 15 wt. %.

20. The perfuming composition of claim 12, wherein the oil phase does not comprise more than 20 wt. % of dipropylene glycol (DIPG).

21. The perfuming composition of claim 20, wherein the oil phase does not comprise more than 10 wt. % of dipropylene glycol (DIPG).
